SEQUENCE LISTING (6 sequences)

SEQ ID NO: 1 - protein, 331 aa, Mus musculus
MHYCVLSTFLLLHLVPVALSLSTCSTLDMDQFMRKRIEAIRGQILSKLKLTSPPEDYPEPDEVPPEVISIYNSTRDLLQEKASRRAAACERERSEQEYYAKEVYKIDMPSHLPSENAIPPTFYRPYFRIVRFDVSTMEKNASNLVKAEFRVFRLQNPKARVAEQRIELYQILKSKDLTSPTQRYIDSKVVKTRAEGEWLSFDVTDAVQEWLHHKDRNLGFKISLHCPCCTFVPSNNYIIPNKSEELEARFADWSHNSPAGGRSALWMLPTALEMCRIIAAFALFTLILRGILDGNGSMNPKGTMLTSVLGHAHIYGVQTLNTPKSSACTTP

SEQ ID NO: 2 - protein, 291 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFADLSHNRPTGGRSVLWMRPIALEMCRIIAAYVHFTLISRGI

SEQ ID NO: 3 - DNA, 993 bp, Mus musculus
atgcactactgtgtgctgagcacctttttgctcctgcatctggtcccggtggcgctcagtctgtctacctgcagcaccctcgacatggatcagtttatgcgcaagaggatcgaggccatccgcgggcagatcctgagcaagctgaagctcaccagccccccggaagactatccggagccggatgaggtccccccggaggtgatttccatctacaacagtaccagggacttactgcaggagaaggcaagccggagggcagccgcctgcgagcgcgagcggagcgagcaggagtactacgccaaggaggtttataaaatcgacatgccgtcccacctcccctccgaaaatgccatcccgcccactttctacagaccctacttcagaatcgtccgctttgatgtctcaacaatggagaaaaatgcttcgaatctggtgaaggcagagttcagggtcttccgcttgcaaaaccccaaagccagagtggccgagcagcggattgaactgtatcagatccttaaatccaaagacttaacatctcccacccagcgctacatcgatagcaaggttgtgaaaaccagagcggagggtgaatggctctccttcgacgtgacagacgctgtgcaggagtggcttcaccacaaagacaggaacctggggtttaaaataagtttacactgcccctgctgtaccttcgtgccgtctaataattacatcatcccgaataaaagcgaagagctcgaggcgagatttgcagactggagtcacaacagtccagccggcggaagaagcgcgctttggatgctgcctactgctttagaaatgtgcaggataattgctgccttcgccctctttacattgattttaagagggatcttggatggaaatggatccatgaacccaaagggtacaatgctaacttctgtgctggggcatgcccatatctatggagttcagacactcaacacaccaaagtcctcagcctgtacaacacca

SEQ ID NO: 4 - DNA, 873 bp, Homo sapiens
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcagacttgagtcacaacagaccaaccggcggaagaagcgtgctttggatgcggcctattgctttagaaatgtgcaggataattgctgcctacgtccactttacattgatttcaagagggatc

SEQ ID NO: 5 - DNA, 1108 bp, Mus musculus
atgcactactgtgtgctgagcacctttttgctcctgcatctggtcccggtggcgctcagtctgtctacctgcagcaccctcgacatggatcagtttatgcgcaagaggatcgaggccatccgcgggcagatcctgagcaagctgaagctcaccagccccccggaagactatccggagccggatgaggtccccccggaggtgatttccatctacaacagtaccagggacttactgcaggagaaggcaagccggagggcagccgcctgcgagcgcgagcggagcgagcaggagtactacgccaaggaggtttataaaatcgacatgccgtcccacctcccctccgaaaatgccatcccgcccactttctacagaccctacttcagaatcgtccgctttgatgtctcaacaatggagaaaaatgcttcgaatctggtgaaggcagagttcagggtcttccgcttgcaaaaccccaaagccagagtggccgagcagcggattgaactgtatcagatccttaaatccaaagacttaacatctcccacccagcgctacatcgatagcaaggttgtgaaaaccagagcggagggtgaatggctctccttcgacgtgacagacgctgtgcaggagtggcttcaccacaaagacaggaacctggggtttaaaataagtttacactgcccctgctgtaccttcgtgccgtctaataattacatcatcccgaataaaagcgaagagctcgaggcgagatttgcaggtattgatggcacctctacatatgccagtggtgatcagaaaactataaagtccactaggaaaaaaaccagtgggaagaccccacatctcctgctaatgttgttgccctcctacagactggagtcacaacagtccagccggcggaagaagcgcgctttggatgctgcctactgctttagaaatgtgcaggataattgctgccttcgccctctttacattgattttaagagggatcttggatggaaatggatccatgaacccaaagggtacaatgctaacttctgtgctggggcatgcccatatctatggagttcagacactcaacacaccaaagtcctcagcctgtacaacacca

SEQ ID NO: 6 - DNA, 988 bp, Homo sapiens
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatc

(19) United States
(12) Patent Application Publication
(10) Pub. No.: US 2008/0200419 A1
(43) Pub. Date: Aug. 21, 2008

(54) Splicing Variant of TGF-beta2 and Uses Thereof

(75) Inventors: Hans-Willem Snoeck, Brooklyn, NY (US); Juhyun Choi, Brooklyn, NY (US)

Correspondence Address: CROWELL & MORING LLP;INTELLECTUAL PROPERTY GROUP, P.O. BOX 14300, WASHINGTON, DC, 20044-4300, US

(73) Assignee: Mount Sinai School of Medicine of New York University, New York, NY (US)

(21) Appl. No.: 12/043,692

(22) Filed: Mar. 6, 2008

Related U.S. Application Data
(63) Continuation of application No. 11/351,953, filed on Feb. 10, 2006, now abandoned.
(60) Provisional application No. 60/652,122, filed on Feb. 11, 2005.

(51) Int. Cl. A61K 48/00; C12N 15/85; C12N 15/867; C12N 5/10; C12N 15/12; C12N 15/861

(52) U.S. Cl. 514/44; 435/455; 536/23.5; 435/372

(57) ABSTRACT

An alternatively spliced form of transforming growth factor-beta2 (TGF-β2), herein denoted Δ6-TGF-β2 is disclosed. Δ6-TGF-β2 differs from TGF-β2 in the sequence of the three C-terminal exons. This novel protein is secreted, induced by cytotoxic stress in hematopoietic stem cells, and specifically blocks the enhancing effects of TGF-β2 on adult stem cells. Δ6-TGF-β2 can be used to protect stem cells from cytotoxic stress, and to enhance maintenance of these cells in vitro during retroviral transduction. In addition, Δ6-TGF-β2 can be used to slow aging and extend longevity.

### CROSS REFERENCE TO RELATED APPLICATION

This application claims the priority of U.S. application Ser. No. 60/652,122 filed Feb. 11, 2005, the disclosure of which is incorporated herein by reference.

### STATEMENT REGARDING FEDERALLY FUNDED RESEARCH

This invention was made with government support under Grant No. RO1AG16723 awarded by the National Institutes of Health. The United States government may have certain rights in this invention.

### TECHNICAL FIELD

The present disclosure relates to a variant of TGF-β2, which is a deletion and frame shift mutation of TGF-β2, and which is capable of protecting hematopoietic and other adult stem cells from cytotoxicity and other stresses.

### INTRODUCTION

The lifelong production of blood cells is maintained by hematopoietic stem cells (HSC) that can give rise to at least eight lineages of mature cells and can self-renew. As they differentiate, HSC progressively lose their self-renewal capacity, and generate primitive multipotential progenitor cells, which become increasingly lineage restricted and give rise in turn to mature cells. HSC are responsible for engraftment after transplantation of bone marrow into a lethally irradiated recipient. Furthermore, because bone marrow is a highly proliferative organ, it is also the first and foremost target of dose-limiting toxicity of chemotherapy for cancer. Finally, as HSC continue to produce blood cells for the lifetime of the individual, they are critical for gene therapy, because they are the ideal targets for gene replacement in a number of genetic diseases that affect the hematopoietic and the immune system. As they are key players in bone marrow transplantation, strategies to enhance hematopoietic recovery after chemotherapy, protect HSC during chemotherapy, expand HSC in vitro and enhance the efficiency of the gene transfer into HSC in the setting of gene therapy, would strongly improve the treatment of leukemia, solid tumors, and genetic disorders affecting the hematopoietic system.

### SUMMARY

A novel extracellular molecule that is induced in HSC after stress and that protects stem cells from stress is described herein. Furthermore, this molecule enhances gene transfer efficiency into stem cells, and maintains or expands stem and progenitor cells in vitro. This molecule beneficially affects aging of the organism, and is involved in lifespan regulation. This molecule, designated herein as Δ6-TGF-β2, is an alternative splice variant of transforming growth beta-2 (TGF-β2) that specifically antagonizes the effects of TGF-β2 on stem cells exemplified herein by early hematopoietic stem and progenitor cells.

In one aspect, there is provided a method of protecting stem cells comprising contacting the stem cells with a functional portion of Δ6-TGF-β2. In certain embodiments, the stem cells are protected against a cytotoxic effect caused by contacting the cells with a chemotherapeutic agent. In a preferred embodiment, the stem cells are contacted with the functional portion of Δ6-TGF-β2 prior to contacting the stem cells with a chemotherapeutic agent. The stem cells may be hematopoietic stem cells or other stem cells, for example skin stem cells.

In certain embodiments, the cells may be contacted with a functional portion of Δ6-TGF-β2 by delivering a recombinant vector operably configured to express the functional portion of Δ6-TGF-β2 from the stem cell. In one exemplary embodiment, the cells are also contacted with resveratrol. In another exemplary embodiment, the cells are contacted with a functional portion of Δ6-TGF-β2 directly. In various embodiments, the stem cells can be contacted with the functional portion of Δ6-TGF-β2 ex vivo or in vivo.

In a related aspect, there is provided a method of enhancing HSC maintenance during the transduction of stem cells with a recombinant vector that includes contacting the stem cells with a functional portion of Δ6-TGF-β2 during a period of transduction with the recombinant vector.

In another related aspect, there is provided a method to delay aging of an mammal that includes contacting the stem cells with a functional portion of Δ6-TGF-β2 or both. In various embodiments, the mammal is contacted with a vector operably configured to express the functional portion of Δ6-TGF-β2 in a cell. In typical embodiments, the cell secretes the Δ6-TGF-β2 which then contacts the stem cells.

In still another related aspect, there is provided a method for maintaining and expanding hematopoietic stem cells during culture in vitro that includes contacting the stem cells with a functional portion of Δ6-TGF-β2 and optionally resveratrol; and culturing the hematopoietic stem cells in the presence of a cell producing Δ6-TGF-β2 or a functional portion thereof.

In another aspect, there is provided a method for producing recombinant Δ6-TGF-2, or a functional portion thereof, that includes expressing the functional portion of Δ6-TGF-β2 in a cell containing a vector operably configured to express the functional portion of Δ6-TGF-β2 from a nucleic acid sequence encoding the functional portion of Δ6-TGF-β2 and obtaining the Δ6-TGF-β2 from at least one of the cell line and a media into which the cell line secretes the Δ6-TGF-β2. In a typical embodiment, the nucleic acid sequence encoding the functional portion of Δ6-TGF-β2 also encodes amino acid sequences selected to secrete the functional portion Δ6-TGF-β2 from the cell line. The cell line can be any cell line, whether of bacterial, fungal, mammalian, insect or plant origin.

The present invention further provides a method for inducing quiescence in stem cells comprising contacting stem cells with a functional portion of Δ6-TGF-β2.

In another aspect, there is provided a isolated nucleic acid sequence that includes a sequence that encodes a functional portion of Δ6-TGF-β2. In a related aspect, there is provided a cell line that includes said isolated nucleic acid sequence.

In yet another aspect, there is provided a composition that includes an isolated functional portion of Δ6-TGF-β2.

### BRIEF DESCRIPTION OF THE DRAWINGS

FIG. 1 depicts sort windows for the flow cytometric isolation of Lin-Sca 1++kit+cells from mouse bone marrow.

FIG. 2 shows the effect of TGF-β2 in vitro. The graph shows the dose response of TGF-β1 and TGF-β2 on proliferation in 5-day cultures of LSK cells from the mouse strains listed on top of the figure, supported by KL, flt3L and TPO (mean ±SEM, n=3 to 13,).

FIGS. 3a-h show the hematopoietic phenotype of adult Tgfb2^{+/-} mice. FIG. 3a is a flow cytometric analysis of the frequency of lin-Sca1⁺⁺ and LSK cells in bone marrow of Tgfb2^{+/-} mice and wt littermates. Bone marrow was pooled from 3 mice for each genotype. 10⁶ events were recorded. FIG. 3b shows the proliferation and CFC output in pre-CFC assays of LSK cells from Tgfb2^{+/-} mice or wt littermates. Bone marrow was pooled from 3 mice for each genotype in each experiment. FIG. 3c shows competitive repopulation of 2.10⁶ wt or Tgfb2^{+/-} bone marrow cells with 2.10⁶ C57BL/6-derived bone marrow cells in C57BL/6 recipients, expressed as ratio for each individual recipient between wt or Tgfb2^{+/-}-derived (CD45.2) and C57BL/6 competitor-derived (CD45.1+CD45.2+) reconstitution at 12 weeks. FIG. 3d shows the log ratio CD45.2+/CD45.1+CD45.2+ in individual primary, secondary and tertiary recipients in serial repopulation experiments from primary donors reconstituted with Tgfb2^{+/-} or wt bone marrow cells as competing cells. FIG. 3e shows the average Δ(log ratio) for wt and Tgfb2^{+/-} bone marrow cells upon serial transplantation (mean ±SEM, n=9 for each genotype). FIG. 3f shows the fraction of LSK cells and of cells falling outside the LSK window (non-LSK) in S/G2/M phase of the cell cycle in Tgfb2^{+/-} mice and in wt littermates (n=4 experiments using mice from different litters). FIG. 3g shows the log(CD45.2+/CD45.1+CD45.2+) in individual recipients in serial competitive repopulation experiments from primary donors reconstituted with adult bone marrow-derived cells (Tgfb2^{+/-} or wt, CD45.2+) as competing cells, and CD45.1+CD45.2+ C57BL/6-derived bone marrow cells as competitor cells. The primary recipients in this experiment were treated with 5-FU 12 weeks after primary reconstitution and 6 weeks before secondary transplantation. FIG. 3h shows the average Δ(log ratio) for wt and Tgfb2^{+/-} bone marrow cells upon serial transplantation after treatment of primary recipient with 5-FU (mean ±SEM, n=3 for wt, n=6 for Tgfb2^{+/-}).

FIG. 4a shows chemotherapy resistance of Tgfb2^{+/-} mice and depicts a Kaplan-Meier analysis of the survival of Tgfb2^{+/-} and wt mice (backcrossed onto C57BL/6 background) after administration of 5-FU 500 mg/kg IV. FIG. 4b shows peripheral platelet (left panel) and leukocyte (right panel) count after injection of a sublethal dose of 5-FU (150 mg/kg IP) in Tgfb2^{+/-} and wt mice. Mice were bled every two days from the retroorbital sinus. N=3 for each day of analysis. FIG. 4c shows a representative example of cell cycle analysis on PFA/Triton-fixed bone marrow cells from Tgfb2^{+/-} and wt mice stained with antibodies and Hoechst 33342 (representative of 3 experiments, P=0.01).

FIG. 5 shows the effect of addition of untreated FCS (serum-containing media, SCM), of FCS treated with proteinase K followed by heat-inactivation at 95° C. (SCM prot. K+HI) or of FCS after dialysis with a cut off of 3.5 kD on the TGF-β2 dose response on the proliferation of LSK cells in serum-free media (SFM) supported by KL, flt3L and TPO (n=4, * significantly different from SFM).

FIG. 6 shows the effect of addition of serum from old and young mice, on the TGF-β2 dose response on the proliferation of LSK cells from old and young mice supported by KL, flt3L and TPO (n=4, * significantly different from SFM).

FIGS. 7a-d depict Tie2 regulation by TGF-β2. FIG. 7a shows the effect of TGF-01 and TGF-β2 on Tie2 expression in purified LSK cells. FIG. 7b depicts flow cytometric analysis of the effect of TGF-β2 on Tie2 expression in serum-free (upper panel) and serum-containing media (lower panel). FIG. 7c shows the expression of Tie2 in LSK cells from Tgfb2^{+/-} mice compared to LSK cells from wt littermates as measured by RT PCR and by flow cytometry. FIG. 7d shows the effect of Ang-1 on the expression TGF-β2 in LSK cells.

FIG. 8 shows the exon structure of TGF-β2 and Δ6-TGF-β2 mRNA (upper panel), and protein sequence starting from the alternative 3′ splice site in TGF-β2 and Δ6-TGF-β2 (lower panel).

FIGS. 9a-e depict the expression of Δ6-TGF-β2. FIG. 9a shows the expression of Δ6-TGF-β2 in LSK cells from 8-week-old and 18-month-old C57BL/6 mice. FIG. 9b shows expression of Δ6-TGF-β2 in lineage positive bone marrow cells from 8-week-old and 18-month-old C57BL/6 mice. FIG. 9c shows expression of Δ6-TGF-β2 in LSK cells from C57BL/6 mice exposed to either 5-FU (150 mg/kg IP) or γ-irradiation (950 cG). FIG. 9d shows expression of Δ6-TGF-β2 in LSK cells from C57BL/6 mice exposed in vitro to H₂O₂ (50 μM for 1 hour) or H₂O₂ and TGF-β2 (1 μg/ml). FIG. 9e shows the effect of cycloheximede (100 μg/ml) on the expression of Δ6-TGF-β2 in LSK cells exposed in vitro to H₂O₂ (50 μM for 1 hour) or H₂O₂ and TGF-β2 (1 pg/ml).

FIGS. 10a-d show the effect of overexpression of Δ6-TGF-β2 in LSK cells. FIG. 10a shows expression levels of TGF-β2 and Δ6-TGF-β2 in LSK cells after transduction with retroviral vectors expressing GFP, full length TGF-β2 and GFP or Δ6-TGF-β2 and GFP. FIG. 10b depicts the dose response of TGF-β2 on the proliferation of LSK cells supported by early-acting cytokines (KL, fltL, TPO) after transduction with retroviral vectors shown on top (n=3, mean ±SEM, *=significantly different from control vector). FIG. 10c shows the dose response of TGF-β1 on the proliferation of LSK cells supported by early-acting cytokines (KL, fltL, TPO) after transduction with retroviral vectors shown on top (n=3, mean ±SEM, *=significantly different from control vector). FIG. 10d depicts expression of Tie-2 LSK cells overexpressing Δ6-TGF-β2.

FIGS. 11a-c show the effect of Δ6-TGF-β2 on OP9 supported cultures. FIG. 11a shows the number of CAFC generated in OP9/Δ6-TGF-β2 cultures from LSK cells relative to number of CAFC generated on control OP9/GFP cells. FIG. 11b shows the survival of lethally irradiated mice after injection of 5.10⁴ cells from OP9/Δ6-TGF-β2 or OP9-GFP supported cultures of LSK cells. FIG. 11c shows a limited dilution analysis of repopulating HSC after 7 days of culture of LSK cells on OP9/Δ6-TGF-β2 or OP9/GFP cells.

FIGS. 12a and b show the competitive repopulation capacity of Δ6-TGF-β2 expressing HSC. FIG. 12a illustrates the experimental strategy. FIG. 12b shows the log ratios in primary transplantations. FIG. 12c shows the log ratio in secondary transformations. Each data point in the secondary recipients represents the average of three mice.

FIG. 13 illustrates the intracellular location of C- and N-terminal fusions of GFP and either full length TGF-β2 or Δ6-TGF-β2.

FIG. 14 shows the effect of soluble Δ6-TGF-β2 on the TGF-β2 dose response by depicting the TGF-β2 dose response on the proliferation of LSK cells supported by KL, flt3L and TPO in the presence of Δ6-TGF-β2 GFP fusion protein or control supernatant (n=3, * significantly different from control).

FIG. 15 demonstrates the involvement of TGF-RII in TGF-β2 signaling in LSK cells by depicting the dose response of TGF-β2 on the proliferation of LSK cells supported by KL, flt3L and TPO from floxed Tgfb2^{+/-} mice treated with PBS or polyl:C (n=3).

FIG. 16 illustrates the regulation of Δ6-TGF-β2 induction by the Sirt1 blocker nicotamide, and the Sirt1 activating compound, resveratrol.

FIG. 17 illustrates the regulation of induction of Δ6-TGF-β2 in embryonic stem cells and in embryonic fibroblasts.

FIG. 18 shows the correlation between the rate of thymic involution (as determined by Hsu et al.) and the frequency of LSK cells in the bone marrow (left) and their to TGF-β2 (right).

FIG. 19 shows the thymus weight in Tgfb2^{+/-} mice and wt littermates at various ages. Each pair of data points represents the average of the 2 to 4 Tgfb2^{+/-} and wt members of a litter.

FIGS. 20a and b show the fraction of naïve (CD44lowCD45RB+) cells of the CD4 and CD8 populations in the peripheral blood of 8-week-old (20a) and 12-month-old (20b) Tgfb2^{+/-} mice and wt littermates.

FIGS. 21a and b show the effect of stem cell genotype on thymic involution. FIG. 21 a shows thymic cellularity 12 months after reciprocal transplants between wt and Tgfb2^{+/-} mice (upper panel). FIG. 21b shows the same analysis, but after stratification of the data according to donor genotype irrespective of recipient genotype and vice versa. (mean ±SEM)

FIG. 22 shows the effect of thymus size on naïve T cell frequency post transplant by depicting the correlation between the fraction of naïve (CD44lowCD45RB+) CD4 cells in the peripheral blood and thymic cellularity in the transplant recipients of FIGS. 21a and b for which data were available (n=10).

FIG. 23a shows the nucleic acid sequence encoding mouse Δ6-TGF-β2. FIG. 23b shows the amino acid sequence of mouse Δ6-TGF-β2 protein.

FIG. 24a shows the nucleic acid sequence encoding human Δ6-TGF-β2 protein.

FIG. 24b shows the amino acid sequence of human Δ6-TGF-β2.

FIG. 25 is a sequence comparison of mouse and human Δ6-TGF-β2 proteins.

### DETAILED DESCRIPTION

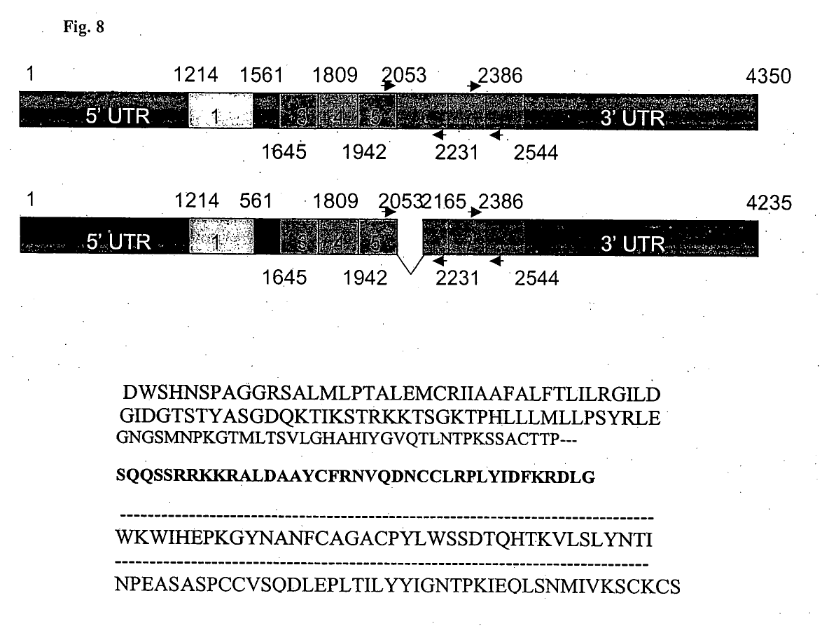

The TGF-β family of cytokines including mammalian isoforms TGF-β1, TGF-β2 and TGF-β3 is well known in the art. See, e.g., Clark et al. (1998) Int. J. Biochem. Cell Biol. 30:293-298. A novel variant of TGF-β2 has been discovered in accordance with the present invention. TGF-β2 mRNA has eight exons. The splice variant discovered herein uses an alternative and downstream 3′ splice site in exon 6 and therefore lacks the 5′ 115 nucleotides of exon 6. This causes a frameshift and premature stop codon after 97 amino acids, as depicted in FIG. 8. The splice variant is referred to herein as Δ6-TGF-β2.

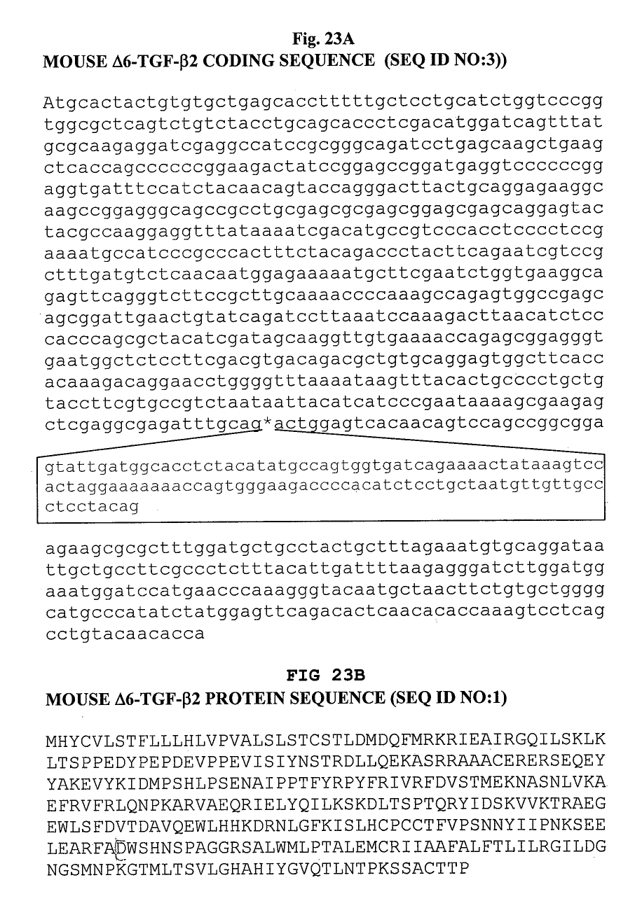

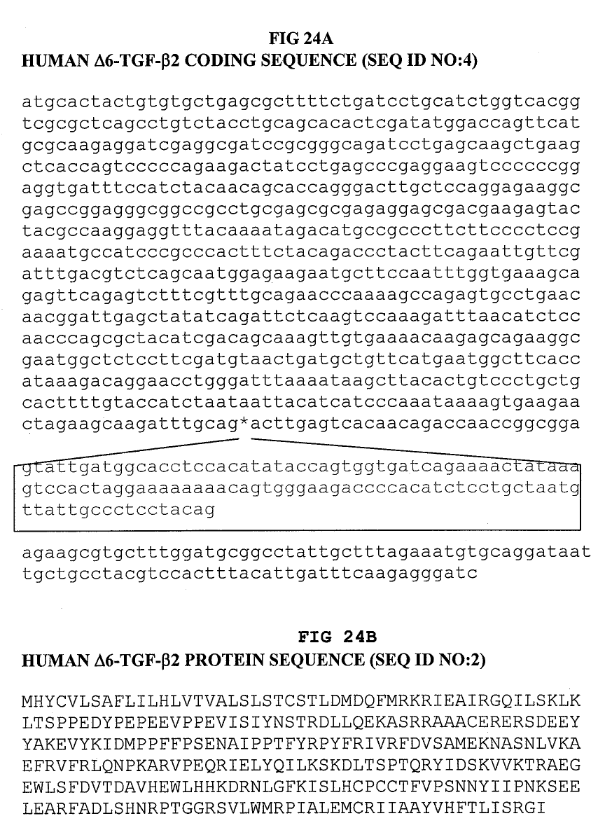

FIG. 23a shows the nucleic acid sequence encoding the mouse Δ6-TGF-β2 protein (SEQ. ID NO: 3). This Figure shows where the sequence is spliced out compared to full length TGF-β2 as indicated by the framing. The alternative splice joint causing the frameshift and deletion is indicated by an asterisk. FIG. 23b shows the amino acid sequence of mouse Δ6-TGF-β2 (SEQ. ID NO: 1).

The inventors have also discovered that Δ6-TGF-β2 is not exclusive to mice. Orthologues of Δ6-TGF-β2 are present in other mammalian systems. FIG. 24a shows a nucleic acid sequence encoding a human orthologue of the mouse Δ6-TGF-β2 that was identified by the inventors (SEQ. ID NO: 4). The sequence encoding human Δ6-TGF-β2 also occurs as result of an alternative splicing of the full length human TGF-β2 as indicated by the framing. Again, the alternative splice point indicated by the asterisks, leads to a frameshift and deletion variant. The alternative splicing point between the human sequence and mouse sequences occurs in corresponding exons between the two species (i.e., exons encoding corresponding amino acids between the two-sequences). FIG. 24b shows the human Δ6-TGF-β2 protein sequence (SEQ. ID NO: 2) resulting from the alternative splicing.

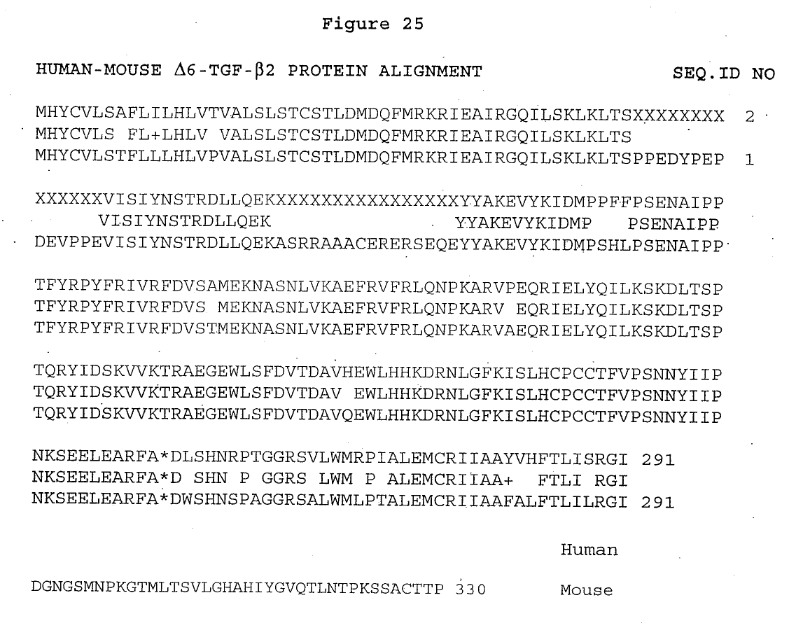

FIG. 25 shows a comparison between the mouse and human amino acid sequences encoded by the mouse and human orthologues of Δ6-TGF-β2. Remarkably, for a splicing variant that requires both a deletion and a frame shift to yield a functional Δ6-TGF-β2 protein, the two sequences show 77% identity in the first 40 corresponding amino acids resulting from the frame shift, with only 10 substitutions, five of which are conservative substitutions in terms of amino acid type. When conservative substitutions are taken into consideration as a measure of amino acid similarity, (e.g., homology) the first 40 amino acids are at least 89% similar. Such conservation in amino acid sequence in an RNA splicing variant between two species indicates that the function of the resulting variant protein is also conserved. The most notable distinction between the human and the mouse Δ6-TGF-β2 protein sequences is that the C-portion of the mouse sequence terminates 77 amino acids after the frame shift, while C-portion of the human sequence terminates 40 amino acids after the frame shift. Accordingly, without being bound by theory, it is believed that no more than the first 40 amino acids from the beginning of the frame shift are needed to confer the novel anti-TGF-β2 activity and resulting affects of Δ6-TGF-β2 on stem cell protection, quiescent maintenance, suppressed proliferation and anti-aging. Alternatively, the novel activity of the Δ6-TGF-β2 may be conferred by the N-terminal, with the first 40 amino acids of the frameshift conferring stability and or intracellular targeting (e.g., to the Golgi).

Using routine experimentation, one of ordinary skill in the art can use standard proteolysis techniques, and/or routine genetic deletion and mutagenesis techniques to define a functional portion and necessary residues of the Δ6-TGF-β2 proteins disclosed herein, as well as orthologues of the same.

Therefore, the compositions and methods provided herein include orthologues of the Δ6-TGF-β2 proteins and nucleic acids encoding the same. Methods of identifying orthologues are known in the art. Normally, orthologues in different species retain the same function due to presence of one or more protein motifs and/or 3-dimensional structures. In evolution, when a gene duplication event follows speciation, a single gene in one species, such as mouse, may correspond to multiple genes (paralogs) in another. As used herein, the term “orthologs” encompasses paralogs. When sequence data is available for a particular species, orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:1204-1210). Programs for multiple sequence alignment, such as CLUSTAL (Thompson J D et al, 1994, Nucleic Acids Res 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs.

Accordingly, as used herein, “a Δ6-TGF-β2 protein” or merely to “Δ6-TGF-β2” means any protein (i.e. polypeptide) includes orthologues, i.e., polypeptides having an amino acid sequence, which in various embodiments, comprises a polypeptide with at least 50% or 60% identity to the mouse or human Δ6-TGF-β2 sequence of SEQ ID NO: 1 or 2. In typical embodiments, a Δ6-TGF-β2 protein has at least 70%, 80%, 85%, 90% or 95% or more sequence identity to the mouse or human Δ6-TGF-β2 sequence of SEQ ID NO: 1 or 2. In other embodiments, a Δ6-TGF-β2 protein includes any protein having at least 50%, 60%, 70%, 80%, 85%, 90% or 95% or more sequence identity from the N-terminus through the first 40 amino acids occurring after the frame shift of the mouse or human Δ6-TGF-β2 sequence of SEQ ID NO: 1 or 2. In yet another embodiment, a Δ6-TGF-β2 protein comprises a polypeptide sequence with at least 50%, 60%, 70%, 80%, or 90% identity to the polypeptide sequence of SEQ ID NO 1 or :2 over the entire length of the mouse or human sequence. All such proteins would be considered orthologues.

As used herein, a “functional portion of Δ6-TGF-β2,” is any portion of a Δ6-TGF-β2 protein, including deletions, additions or amino acid substitutions thereof, that confers protection from cytotoxic effects and/or has the other anti-TGF-β2 affects described herein, including quiescent maintenance, suppressed proliferation and/or anti-aging affects. In typical embodiments, a Δ6-TGF-β2 protein comprises a polypeptide sequence with at least 50%, 60%, 70%, 80%, 85%, 90% or 95% or more sequence identity to a functional portion of the polypeptide presented in SEQ ID NO 1 or :2.

Similarly, as used herein, “a nucleic acid sequencing encoding a functional portion of Δ6-TGF-β2” is a nucleic acid that encodes a protein sequence that is at least 50% to 60% identical over its entire length to mouse or human Δ6-TGF-β2 sequence of SEQ ID NO: 1 or 2, or nucleic acid sequences that are complementary to the same. In typical embodiments, the nucleic acid encodes a polypeptide having least 70%, 80%, 85%, 90% or 95% or more sequence identity to the mouse or human Δ6-TGF-β2 sequence of SEQ ID NO: 1 or 2 or a functionally portion thereof, or complementary sequences to the coding sequences. In other embodiments the nucleic acid sequence encodes a protein having other embodiments, a Δ6-TGF-β2 protein includes any protein having at least 50%, 60%, 70%, 80%, 85%, 90% or 95% or more sequence identity from the N-terminus through the first 40 amino acids occurring after the frame shift of the mouse or human Δ6-TGF-β2 sequence of SEQ ID NO: 1 or 2. In yet other embodiments the nucleic acid sequence encodes a protein having at least 50%, 60%, 70%, 80%, 85%, 90% or 95% or more sequence identity to a functional portion of the mouse or human Δ6-TGF-β2 sequence of SEQ ID NO: 1 or 2.

Because the Δ6-TGF-β2 proteins described herein are alternative splice variants of a known genomic sequence that encodes TGF-β2, typical embodiments of nucleic acids provided herein are recombinantly engineered nucleic acids particularly configured with operable sequences to express Δ6-TGF-β2 rather than TGF-β2. In this context, nucleic acids encoding Δ6-TGF-β2 typically mean nucleic acids having cDNA or RNA sequences engineered without introns to specifically express Δ6-TGF-β2. However, various other embodiments also include isolated nucleic acids that encode the genomic sequence, provided that the such embodiments are particularly engineered with operable modifications designed to over express the Δ6-TGF-β2 variant relative to the amount of TGF-β2 that would ordinarily expressed without the modifications. Such embodiments may include, for example, splice junctions that are engineered to preferentially form an mRNA encoding the Δ6-TGF-β2 protein rather than TGF-β

As used herein, “percent (%) sequence identity” with respect to a specified subject sequence, or a specified portion thereof, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1997) 215:403-410; website at blast.wustl.edu/blast/README.html) with search parameters set to default values. The HSP S and HSP S2 parameters are dynamic values and are established by the program itself depending upon the composition of the particular sequence and composition of the particular database against which the sequence of interest is being searched. A “% identity value” is determined by the number of matching identical nucleotides or amino acids divided by the sequence length for which the percent identity is being reported.

“Percent (%) amino acid sequence similarity” is determined by doing the same calculation as for determining % amino acid sequence identity, but including conservative amino acid substitutions in addition to identical amino acids in the computation. A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

Thus, in certain embodiments, a Δ6-TGF-β2 polypeptide (or nucleic acid encoding the same) also includes a polypeptide having at least 50%, 60%, 70%, 75%, 80%, 85%, 90%, 95% or more sequence similarity to a functional portion of the mouse or human Δ6-TGF-β2 polypeptides of SEQ ID NO: 1 or 2.

As a result of the degeneracy of the genetic code, a number of nucleic acid sequences encoding an Δ6-TGF-β2 polypeptide can be produced. For example, codons may be selected to increase the rate at which expression of the polypeptide occurs in a particular host species, in accordance with the optimum codon usage dictated by the particular host organism (see, e.g., Nakamura et al, 1999). Such sequence variants may be used in the methods of this invention.

Δ6-TGF-β2 proteins can be isolated from any cell line, including bacterial, fungal, mammalian, insect or plant cell lines carrying an expression vector operably configured to express the Δ6-TGF-β2 protein. In a typical practice, the vector may contain a “HIS tagged” sequence commonly used in the art. A HIS tag sequence is a nucleic acid sequence that is part of a vector that encodes poly histidine residues configured in the vector to produce an in-frame fusion protein containing the poly histidine residues fused to an end of the desired sequence to be expressed. The poly histidine residues facilitate isolation of the protein by reversible binding of the poly histidine to a substrate containing nickel.

In another approach, antibodies that specifically bind known Δ6-TGF-β2 polypeptides are used for ortholog isolation (see, e.g., Harlow and Lane, 1988, 1999). Western blot analysis can determine that an Δ6-TGF-β2 orthologue (is present in a crude extract of a stem cell line of a given species or is secreted into a media from the cell line. When reactivity is observed, the cDNA sequence encoding the candidate ortholog may be isolated by screening expression libraries representing the particular species. Expression libraries can be constructed in a variety of commercially available vectors, including lambda gt11, as described in Sambrook, et al., 1989. Once the candidate ortholog(s) are identified by any of these means, candidate orthologous sequences are used as bait (the “query”) for the reverse BLAST analysis against sequences from mouse or human Δ6-TGF-β2 or other species in which a Δ6-TGF-β2 polypeptide has been identified.

TGF-β2 is a positive regulator of HSC, which makes these cells more vulnerable to cytotoxic stress, but at the same time enhances the regenerative capacity of these cells. Δ6-TGF-β2 is induced by stress, including oxidative stress, irradiation and cytotoxic agents, and specifically blocks the enhancing effects of TGF-β2 on HSC, thereby protecting these cells from stress. After stress has abated, induction of Δ6-TGF-β2 decreases, and TGF-β2 signaling is allowed to resume, further repressing Δ6-TGF-β2, and enhancing regenerative capacity of stem cells for tissue repair. Thus, this system will protect HSC during stress, and will enhance their regenerative capacity after stress.

Δ6-TGF-β2 can be produced as a stable, recombinant protein by methods well-known to those of ordinary skill in the art, and has utilities including the following.

Δ6-TGF-β2 is useful for the protection of stem cells from stress, for example, cytotoxic stress caused by chemotherapy and radiation for cancer therapy. Δ6-TGF-β2 is a secreted, soluble molecule that has a highly specific effect on stem cells, and, as far as is known, not on other cells. Therapy with Δ6-TGF-β2 thus specifically targets stem cells including skin and HSC. Use of Δ6-TGF-β2 is preferable to, for example, neutralizing TGF-β2 antibodies, since the latter will block all effects of TGF-β2 on any cell in the body, whereas the former only blocks the specific effect of TGF-β2 on stem cells that makes these more vulnerable during stress.

Accordingly, the present invention provides a method of protecting stem cells comprising contacting stem cells in need of protection with a functional portion of Δ6-TGF-β2. The cells may be contacted with the functional portion of Δ6-TGF-β2 directly or with a vector configured to express the functional portion of Δ6-TGF-β2. The stem cells may further be contacted with resveratrol. In a preferred embodiment the stem cells are HSC. In another preferred embodiment the stem cells are protected from stress caused by chemotherapy or radiation therapy. The stem cells may be ex vivo or in vivo. For in vivo methods, those of skill in the can determine formulations and dosages depending upon means of administration, target site, and other considerations with reference, for example, to Gilman et al. (1990) The Pharmaceutical Basis of Therapeutics (9^{th} Ed.), Perganon Press and Remington's Pharmaceutical Sciences (17th Ed.) Mack Publishing Co., Easton, Pa.

Δ6-TGF-β2 is also useful for the enhancement of transduction of HSC. HSC retain their repopulation capacity after transduction with Δ6-TGF-β2 better than after transduction with control vectors. The likely reason is that Δ6-TGF-β2, once expressed in HSC, contributes to the maintenance of HSC in culture during the transduction period. Transduction of HSC is currently, together with bone marrow transplantation, the only potential therapy for a number lethal genetic diseases affecting the hematopoietic and immune system. Use of recombinant Δ6-TGF-β2 to enhance HSC maintenance during ex vivo transduction strongly increases the clinical applicability of this type of therapy. Accordingly, the present invention provides a method of enhancing HSC maintenance during transduction of HSC comprising contacting HSC undergoing transduction with a functional portion of Δ6-TGF-β2.

The present invention also provides a method for the expansion of radioprotecting and long-term repopulating stem cells in vitro comprising contacting such cells with a functional portion of Δ6-TGF-β2.

Quiescence of stem cells is associated with delayed thymic involution and longer life span in mice. Administration of Δ6-TGF-β2 therefore contributes to life span extension by maintaining stem cells in quiescence and preventing premature functional decline of these cells. The present invention provides a method for inducing quiescence in stem cells comprising contacting stem cells with a functional portion of Δ6-TGF-β2.

One method of delaying aging is to administer Δ6-TGF-β2 or a functional portion thereof systemically to the mammal, for example, by injection. Another method is to use vector based delivery, which includes transforming a cell within the mammal with a vector operably configured to express the functional portion of Δ6-TGF-β2 from the cell. The Δ6-TGF-β2 is secreted from the transformed cell and contacts the stem cells systemically. In typical embodiments, the transduced cell will be a liver cell, which may or may not be a hematopoietic stem cell. Vectors and other procedures for gene therapy, especially using liver cells as the host cell for transduction are well known in the art. Typical vectors include engineered retroviruses, adenovirus and adeno-associated viruses. The retroviruses may be, for example, a lentivins. Transduction can also be accomplished using other techniques known in the art, such as by naked DNA transfer or transfer mediated by encapsulating the nucleic acid in a cationic lipid vesicle. Transduction may be accomplished by direct in-vivo administration, or by performing the transduction ex-vivo and then transferring the transduced cells back into the organism. Many suitable vectors and other methods of transduction have been described in the art including in various patents and patent applications, which are incorporated herein by reference to the extent necessary to teach one of ordinary skill in the art how to transduce a cell with an exogenous nucleic acid sequence and introduce the cell back into the organism.

From the foregoing it will be appreciated that, although specific embodiments of the invention have been described herein for purposes of illustration, various modifications may be made without deviating from the spirit and scope of the invention.

All references cited herein are incorporated herein in their entirety.

The following non-liming examples serve to further illustrate the present invention.

### EXAMPLE 1

### TGF-β2 is a Positive Regulator of HSC Cycling and Function

A. TGF-β2 is a Genetically Determined Positive, Regulator of HSC

The kinetic behavior of the hematopoietic stem and progenitor cell compartment shows mouse strain-dependent variation in inbred mice, and are determined by multiple loci. These are therefore quantitative traits. The study of the identity of these quantitative traits, and of their biological significance is a focus of the inventors' investigations.

The inventors have shown previously that the frequency of hematopoietic stem and progenitor cells as determined by the lin-Sca1++kit+ (LSK) phenotype, as well the response of these cells to early-acting cytokines shows wide mouse strain-dependent variation. Transforming growth factor-beta (TGF-β) is considered a negative regulator of hematopoietic stem and progenitor cells. The inventors investigated whether there was also quantitative genetic variation in the responsiveness of LSK cells to TGF-β. As there are three isoforms of TGF-β (TGF-β1, -β2 and -β3), which are encoded on different chromosomes, the inventors tested the effects of all three isoforms. The data have been published and are summarized here.

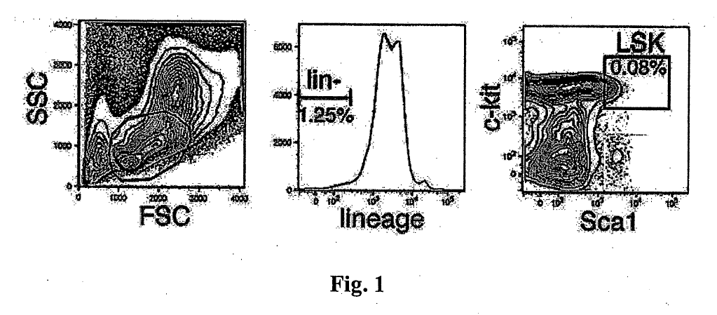

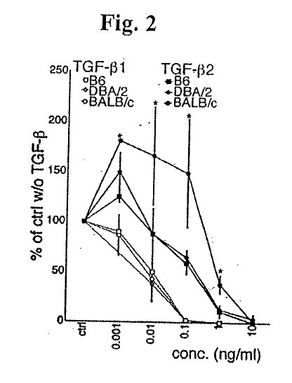

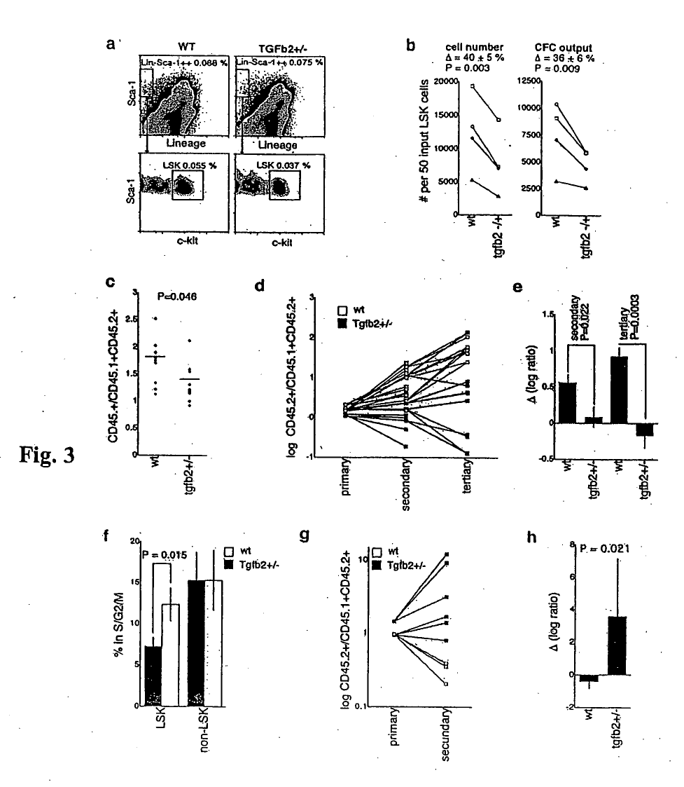

In vitro, TGF-β2 had a biphasic dose response with a stimulatory effect at low concentrations on the proliferation of purified LSK cells (FIG. 1) in response to the early acting cytokines kit ligand (KL), flt3 ligand (flt3L) and thrombopoietin (TPO) (FIG. 2). In contrast, the dose responses of TGF-β1 (FIG. 2) and -β3 (not shown) were inhibitory without a stimulatory effect at low concentrations. The dose response of TGF-β2 was subject to mouse strain-dependent variation (FIG. 2), which mapped to a locus on chromosome 4. This locus overlaps with a quantitative trait locus on chromosome 4 regulating the frequency of LSK cells, suggesting that TGF-β2 is a regulator of LSK cells in vivo. Studies in knockout mice revealed that the stimulatory effect of TGF-β2 on the proliferation of LSK cells, observed at low TGF-β2 concentrations in vitro, is relevant in vivo. The frequency of LSK cells, their proliferative capacity in vitro, as well as the cycling activity and the serial repopulating capacity of HSC were lower in adult Tgfb2^{+/-} mice than in wt littermates (FIG. 3). Importantly, Tgfb2^{+/-} HSC are protected from cell cycle-specific cytotoxic agents. This is proven by the finding that in mice competitively repopulated with wt and Tgfb2^{+/-} bone marrow, injection of 5-FU, a cytotoxic chemotherapeutic agent that kills dividing cells, preferentially affects the subsequent repopulation capacity of wt HSC, while Tgfb2^{+/-} HSC were protected (FIG. 3g, 3h). The latter data indicates that TGF-β2 directly enhances the self-renewing cycling activity of HSC. Furthermore, as even in a wt environment, Tgfb2^{+/-} HSC cycle more slowly than wt HSC, it is likely that TGF-β2 acts at least in part cell autonomously.

Tgfb2^{+/-} mice were tested to determine whether they were more resistant than wt mice to 5-FU, a cytotoxic chemotherapeutic agent that kills dividing cells. As mice can be rescued from lethal doses of 5-FU by bone marrow transplantation, the lethal effects of 5-FU are due to hematopoietic failure. Consistent with the protection of Tgfb2^{+/-} HSC from 5-FU toxicity, the survival of Tgfb2^{+/-} mice after IV injection of 500 mg/kg 5-FU was significantly better than that of wt mice (P<0.0001) (FIG. 4a). A sublethal dose of 5-FU (150 mg/kg IP) was administered to Tgfb2^{+/-} and wt mice. In this case, the degree of leukopenia and thrombocytopenia as well as the rate of recovery were similar in Tgfb2^{+/-} and wt mice (FIG. 4b). Together, these data indicate that TGF-β2 is a critical regulator of the cycling of HSC and of the lethality of cytotoxic agents. As the biological effects of sublethal doses of 5-FU were similar in Tgfb2^{+/-} and wt mice, these data indicate that TGF-β2 deficiency does not affect the metabolism of 5-FU.

Taken together, the foregoing data prove that TGF-β2 plays a critical role in the biology of HSC, in particular in the regulation of the cycling activity of HSC. While deficiency of TGF-β2 is probably without consequence in steady state, a phenotype consistent with decreased cycling activity becomes obvious in conditions of stress. In the setting of serial transplantation, this decreased cycling activity results in a progressive decline in serial competitive repopulation capacity compared to ‘normally’ cycling wt HSC, whereas during administration of cytotoxic agents, this results in enhanced protection. The proliferative effect of TGF-β2 is specific for the HSC compartment. TGF-β2 responsiveness is also subject to mouse strain-dependent variation and may play a role in the quantitative genetic variation in the hematopoietic stem cell compartment.

B. The Mechanisms of Action of TGF-β2

1. Serum Factor

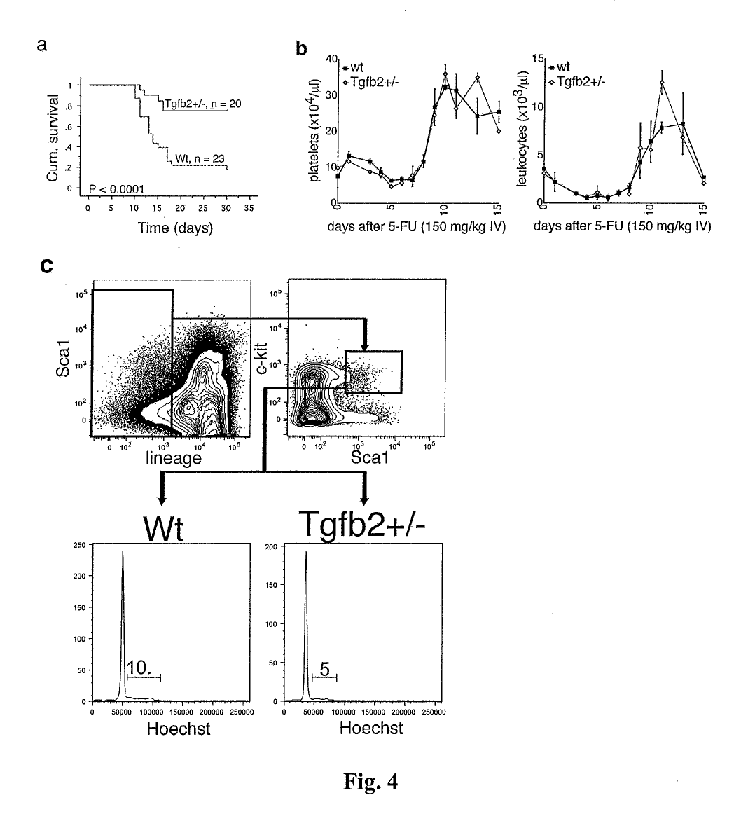

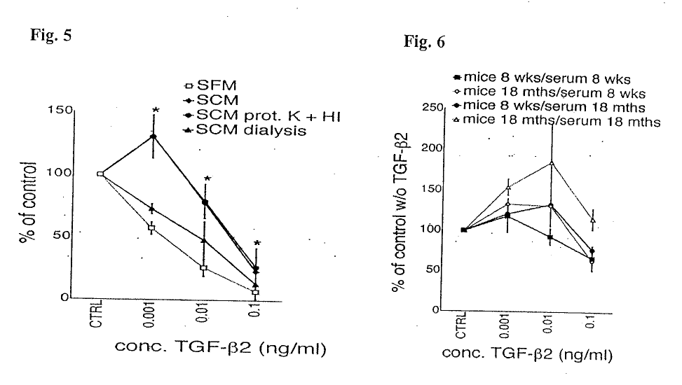

A serum factor is required for the stimulatory effect of low concentrations of TGF-β2 on the proliferation of LSK cells. In the absence of serum, TGF-β2 behaves exactly like TGF-β1, and is a potent inhibitor of LSK cell proliferation (FIG. 5). Initial biochemical analysis has revealed that this serum factor is not a protein as it is resistant to proteinase K and heat treatment (boiling for 10 minutes), and as serum can be depleted from this activity by dialysis, using membranes with a cut-off of 3 kD (FIG. 5). Furthermore, this factor is also present in mouse serum, and the activity of this factor is higher in serum from old mice than in serum from young mice (FIG. 6), implicating stimulatory TGF-β2 signaling in stem and progenitor cells in the regulation of the function of stem cells that have undergone replicative or other forms of stress.

2. Regulation of Tie2 Expression

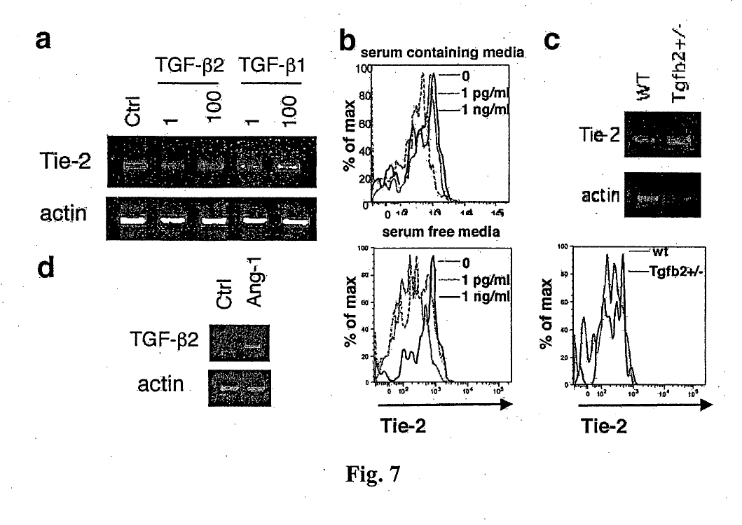

TGF-β2 not only has a proliferative effect on HSC, but also affects the interaction of HSC with a critical cell in the microenvironment in vivo, the osteoblast. Osteoblasts produce Angiopoietin-1 (Ang-1), which signals through the Tie-2 receptor expressed on HSC to keep the latter in quiescence. As measured by semiquantitative PCR, low concentrations of TGF-β2 (1 pg/ml), but not of TGF-β1, decreased the expression of Tie-2, whereas higher concentrations had no detectable effect (FIG. 7a). The finding that TGF-β2 regulates the expression of Tie-2 in LSK cells was confirmed by flow cytometry: addition of 1 pg/ml of TGF-β2 to LSK cells decreased the expression of Tie-2, whereas 1 ng/ml had no effect (FIG. 7b). Similar to the proliferative effects of TGF-β2 on LSK cells, the repressive effect of TGF-β2 on Tie-2 expression was dependent on serum: in serum-free media, TGF-β2 induced a dose-dependent increase in the expression of Tie-2 (FIG. 7b). Thus, both the proliferative effects of TGF-β2 on LSK cells and its effect on Tie-2 expression are biphasic and serum-dependent. In LSK cells from Tgfb2^{+/-} mice (backcrossed onto the C57BL/6 background for 11 generations), Tie-2 expression was higher than in LSK cells from wt littermates, again demonstrating regulation of Tie2 expression by TGF-β2. As Tie2+ HSC are more quiescent, these data are consistent with the quiescence of HSC in Tgfb2^{+/-} mice. Furthermore, as Tgfb2^{+/-} HSC as well as Tgfb2^{+/-} mice are protected from 5-FU toxicity, these data are also consistent with the finding of Arai et al. that Tie2+ HSC are resistant to 5-FU. Addition of Ang-1 to LSK cells indeed upregulated the expression of TGF-β2. These data indicate that there is a balance between the biphasic effect of TGF-β2 on the expression of Tie-2, and the stimulatory effect of Tie-2 signaling on the expression of TGF-β2.

3. Conclusions from the Inventors' Studies

TGF-β2 is specific positive regulator of the cycling activity of HSC in vivo, both through a direct proliferative effect on HSC and through down regulation of the expression of the Tie2 receptor on HSC. As such, TGF-β2 enhances the regenerative capacity of HSC, but also makes these cells more sensitive to cytotoxic agents.

### EXAMPLE 2

### An Alternative Splice Form of TGF-β2, Δ6-TGF-β2, is Induced by Stress and Specifically Blocks the Effects of TGF-β2 on Hematopoietic Stem and Progenitor Cells

A. An Alternative Splice Variant of TGF-β2

The inventors discovered a novel splice variant of TGF-β2. TGF-β2 mRNA has 8 exons. This splice variant uses an alternative and downstream 3′ splice site in exon 6 and therefore lacks the 5′ 115 nucleotides of exon 6. This causes a frameshift and premature stop codon after 97 amino acids (FIG. 8). The splice variant is termed Δ6-TGF-β2. The splice variant may be detected using PCR primers spanning exons 5 to 6.

B. Expression Pattern of Δ6-TGF-β2

Several aspects of the expression pattern of Δ6-TGF-β2 are of particular note:

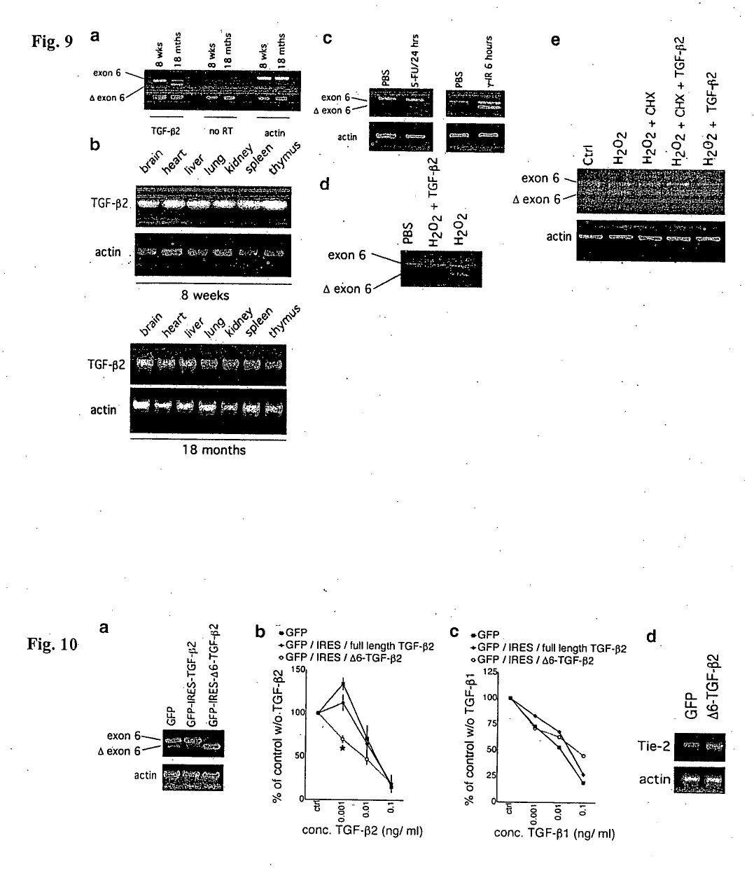

The expression of Δ6-TGF-β2 increases with age in LSK cells from C57BL/6 mice. This phenomenon was only observed in LSK cells and not in mature hematopoietic cells (spleen) or in other tissues (FIGS. 9a and 9b). No such splice form was found for TGF-β1 (not shown).

Stress in vivo and in vitro induces Δ6-TGF-β2. Treatment of mice with the cytotoxic agent 5-FU (150 mg/kg) or lethally irradiation (950 cG) induced Δ6-TGF-β2 LSK cells, demonstrating that hematopoietic stress in vivo induces alternative splicing of TGF-β2 pre-mRNA (FIG. 9c). The stress induced induction of Δ6-TGF-β2 could be reproduced in vitro as exposure of LSK cells to the oxidant H₂0₂ for 1 hour induced Δ6-TGF-β2 (FIG. 9d)

Induction of Δ6-TGF-β2 is blocked by TGF-β2. Addition of TGF-β2 (1 pg/ml) during exposure to H₂O₂ abrogated alternative splicing of TGF-β2 pre-mRNA. Thus, TGF-β2 signaling represses alternative splicing of TGF-β2 pre-mRNA in LSK cells (FIG. 9d).

The induction of alternative splicing of TGF-β2 pre-mRNA may not require protein synthesis but its repression by TGF-β2 does. The rapidity of the induction of Δ6-TGF-β2 suggests that protein synthesis is not involved. Addition of the protein synthesis blocker cycloheximide (100 μg/ml) slightly affected H₂O₂-induced alternative splicing of TGF-β2 pre-mRNA, suggesting that a role for protein synthesis cannot be entirely excluded, but is likely minor. On the other hand, the repressive effect of TGF-β2 on the induction of Δ6-TGF-β2 appeared almost entirely dependent on protein synthesis (FIG. 9e).

C. Role of Δ6-TGF-β2 In Vitro: Antagonism of TGF-β2

1. Overexpression of Δ6-TGF-β2 Affected the TGF-β2 Dose Response, and Increased Tie2 Expression in LSK Cells.

To investigate the role of Δ6-TGF-β2 in long-term repopulating HSC, MSCV-based retroviral constructs were generated that contain GFP or GFP and Δ6-TGF-β2 separated by an internal ribosomal entry site (IRES) sequence. Bone marrow cells were transduced with these retroviral vectors and GFP+lin-Sca1++ cells (c-kit expression decreases on proliferating progenitor and stem cells) were isolated by cell sorting and cultured.

Whereas in control and in full length TGF-β2 overexpressing LSK cells the dose response of TGF-β2 in the presence of serum in vitro was biphasic, with a stimulatory effect at low concentrations, in lin-Sca1++ cells overexpressing Δ6-TGF-β2, no stimulatory effect was detected at low concentrations, and TGF-β2 was a potent inhibitor of proliferation (FIG. 10a,b). Thus, Δ6-TGF-β2 specifically blocked the unique stimulatory effect of TGF-β2 on the proliferation of LSK cells. Neither the inhibitory phase of the dose response nor baseline proliferation in the absence of TGF-β2 were affected. Further supporting the finding that the blocking effect of Δ6-TGF-β2 is specific for TGF-β2, the dose response of TGF-β1 was not affected by overexpression of Δ6-TGF-β2 in LSK cells (FIG. 10c). Entirely consistent with these data, overexpression of Δ6-TGF-β2 strongly upregulated Tie2 expression, again showing that Δ6-TGF-β2 blocked a LSK cell specific, serum-dependent effect of TGF-β2 (FIG. 10d).

As TGF-β2 is a positive regulator of HSC function in vivo, it is likely that Δ6-TGF-β2 will negatively affect HSC function in vivo. Thus, Δ6-TGF-β2 may switch TGF-β2 from a positive to a negative regulator of stem and progenitor cells in vivo, and may drive HSC into quiescence. As Δ6-TGF-β2 is induced by stress in LSK cells, this response may be geared to protect from stress until the stressful insult is over. Δ6-TGF-β2 therefore protects HSC from cytotoxic stress.

2. Effects of Δ6-TGF-β2 In Vitro: Maintenance of HSC and Generation of Radioprotection Capacity

To test the effect of exposure of hematopoietic stem and progenitor cells to Δ6-TGF-β2 in vitro in the context of an environment that mimics the stem cell niche to some extent, OP9 cells stably expressing Δ6-TGF-β2 were generated. OP9 cells are derived from M-CSF-deficient op/op mice, and support the formation of cobblestone areas generated from primitive stem and progenitor cells as well as limited maintenance of HSC. OP9 cells expressing GFP-IRES-Δ6-TGF-β2 (OP9/Δ6-TGF-β2) and control OP9 cells expressing GFP (OP9/GFP) were generated by retroviral transduction followed by isolation of GFP+ cells by flow cytometric cell sorting. 200 to 400 LSK cells were seeded onto these stable cell lines in the presence of KL and IL6. Total cell (not shown) and cobble area-forming cell (CAFC) number (FIG. 11a) after 7 days of culture were five fold higher in the presence of OP9/Δ6-TGF-β2 cells than in the presence of OP9/GFP cells. Cell number expanded on average 1300-fold in OP9/Δ6-TGF-β2 supported cultures. To examine the potential of the cells generated in OP9/Δ6-TGF-β2 and OP9/GFP-supported cultures, 5.10⁴ CD45+ hematopoietic cells harvested from these cultures were injected into lethally irradiated mice. Cells cultured on OP9/Δ6-TGF-β2 cells had significantly more potent radioprotection capacity on a per cell basis compared to cells grown on OP9/GFP cells (FIG. 11b). Radioprotection is provided by committed progenitor cells that are capable of rapidly generating mature cells, and is therefore not a measure of stem cell activity. Nevertheless, in all of the surviving mice, long-term donor-derived multilineage engraftment was observed, ranging from 5% to 100% (not shown). These data indicate that the injected cells contained long-term repopulating stem cells.

To measure stem cell activity, limit dilution competitive repopulation assays were performed (FIG. 11c). Lethally irradiated CD45.1+ mice were injected with 2,500, 5,000, 10,000 and 20,000 CD45.2+ cells harvested after 7 days of culture of LSK cells supported by OP9/Δ6-TGF-β2 or OP9/GFP cells, together with 2.10⁵ CD45.1+CD45.2+ competitor cells. The contribution of donor-derived CD45.2+ cells to the B cell, T cell and myeloid lineages was measured by flow cytometry of peripheral blood cells' stained for CD19, Thy1 and Gr1/Mac1. Mice with a donor contribution of >0.5% in each lineage were considered positive. In parallel, the same experiments were performed with freshly isolated LSK cells before plating. Here, 5, 10, and 20 cells were injected together with 2.10⁵ CD45.1+CD45.2+ competitor cells. The estimated HSC frequency among fresh LSK cells was approximately ⅕ (n=2). The frequency of repopulating HSC was similar in OP9/GFP and in OP9/Δ6-TGF-β2, and was approximately 1/5000 (n=2). However, the total number of cells generated in OP9/Δ6-TGF-β2 supported cultures was consistently 5-fold higher than in OP6/GFP cultures (n=7). Therefore, the stem cell content was approximately 5-fold higher in OP9/Δ6-TGF-β2 than in OP9/GFP cultures. Calculated as the number of HSC per 100 input LSK cells, HSC number was maintained on OP9/Δ6-TGF-β2 cells, but declined 5-fold on OP9/GFP cells. The maintenance of HSC in OP9/Δ6-TGF-β2-supported cultures is consistent with the data obtained after retroviral transduction described in the previous section.

Thus, culture of LSK cells in the presence of OP9/Δ6-TGF-β2, cells resulted in maintenance of HSC together with a strong induction of radioprotection capacity, and a 1300-fold increase in cell number.

3. Effect of Overexpression of Δ6-TGF-β2: Maintenance of HSC In Vitro, but Loss of Serial Repopulation Capacity In Vivo

The effect of overexpression of Δ6-TGF-β2 in competitive repopulation studies was examined. MSCV-based retroviral constructs were generated that contain GFP and Δ6-TGF-β2 separated by an internal ribosomal entry site (IRES) sequence or GFP-IRES (termed GFP hereafter). Bone marrow (day 5 post-5-FU) was stimulated with KL, flt3L and TPO, and transduced using a ‘spinfection’ protocol. 5.10⁵ GFP+ cells were isolated by cell sorting after 48 hours. CD45.1+GFP or CD45.1+GFP-IRES-Δ6-TGF-β2 cells were injected into lethally irradiated CD45.2+ mice together with equal numbers of CD45.1+CD45.2+GFP bone marrow cells (FIG. 12a). In this setup, both CD45.1+GFP control cells and CD45.1+GFP-IRES-Δ6-TGF-β2 cells were competed against the same population of CD45.1+CD45.2+ GFP competitor cells. This approach rules out bias because of the CD45 allelic variant, which we have shown may affect repopulation potential. Furthermore, by selecting for GFP+ cells before transplantation, variation in transduction efficiency is minimized as a confounding variable. To linearize the data, the log (% CD45.1+/% CD451+CD45.2+) was compared between CD45.1+GFP cells and CD45.1+GFP-IRES-Δ6-TGF-β2 cells. Mice were analyzed 15 to 18 weeks after reconstitution. As shown in FIG. 12b, transduction with Δ6-TGF-β2 conferred a repopulation advantage compared to transduction with GFP only. This difference in log(% CD45.1+/% CD451+CD45.2+) represents a 3- to 4-fold difference in repopulation capacity (P=0.05; n=20 mice from 3 independent experiments).

These data suggest increased repopulation capacity of Δ6-TGF-β2 expressing HSC. However, it is also possible that expression and secretion of Δ6-TGF-β2 during the transduction culture enhanced the maintenance of HSC, as repopulating HSC are known to be lost rapidly in these conditions. To further investigate the effect of Δ6-TGF-β2 expression on repopulation capacity in the absence of in vitro culture, serial transplantation using bone marrow from primary recipients was performed. If the enhancing effect of Δ6-TGF-β2 is due to increased maintenance of HSC during in vitro culture, then no further increase in the contribution of CD45.1+GFP-IRES-Δ6-TGF-β2 cells should be observed in secondary recipients. 2.10⁶ bone marrow cells from competitively repopulated recipients were injected into CD45.2+ mice (three secondary recipients per primary recipient, a total of 9 recipients for each group). Three months later, the contribution of CD45.1+ and CD45.1+.CD45.2+ cells to hematopoiesis was assessed. The difference in the log reconstitution ratio in primary and secondary recipients was used for statistical analysis. The reason for this strategy is the following. The difference in the percentage contribution of CD45.1+ cells is not a good measure of any shift in reconstitution capacity in secondary recipients, as a small change around 50% does not reflect a major shift in the function or number of HSC, whereas a small change around 95% actually represents a large shift. A better measure is the ratio between the CD45.1+/CD45.1+CD45.2+ ratio pre (input) and post (output) secondary reconstitution. By analogy with the way ratiometric data are handled in the analysis of microarrays, the difference between log CD45.1+/CD45.1+CD45.2+ ratios in primary and secondary recipients can be used. An advantage of log transformation is that a ratio smaller than 1 will give a negative value, and negative ratios will extend over the same numerical ranges as positive ones (e.g., a ratio of 0.01 gives a log ratio of −2, a ratio of 100 gives a log ratio of +2), thus normalizing the data. Δ(log ratio) values were negative for Δ6-TGF-β2 expressing HSC, indicating that Δ6-TGF-β2-expressing HSC lost reconstitution capacity compared to GFP-expressing control HSC after serial transplantation (FIG. 12c). In contrast, no significant changes in the log ratio were observed in the control mice (FIG. 12c). The difference in Δ(log ratio) between the two types of recipients was statistically significant (P=0.002). These data indicate that the higher repopulation capacity of Δ6-TGF-β2-transduced bone marrow cells is caused by a better maintenance of HSC during transduction, while their intrinsic repopulation capacity is in fact decreased. The expression of GFP was similar in primary and secondary recipients (not shown).

D. Mechanism of Action of Δ6-TGF-β2: Δ6-TGF-β2 is a Secreted Factor

TGF-β is produced as a propeptide consisting of the N-terminal latency-associated peptide (LAP) and the C-terminal active TGF-β. The mRNA consists of 8 exons. Active TGF-β2 starts near the end of exon 6. During intracellular processing, LAP and active TGF-β are cleaved, and remain non-covalently associated. After secretion in this inactive or latent conformation, TGF-β can be activated by heat, acid, chaotropic agents, plasmin and thrombospondin1. Furthermore, LAP can be sequestered through integrin binding, and can bind the mannose-6-phosphate receptor. In Δ6-TGF-β2, the use of an alternative 3′ splice site causes a 115 nucleotide deletion and a frame shift, resulting in a premature stop codon 291 nucleotides downstream. The C-terminal 97 amino acids of Δ6-TGF-β2 are thus different from the C-terminal of full length TGF-β2 (FIG. 8). As Δ6-TGF-β2 cannot produce any active TGF-β2 protein, it is highly unlikely to signal through the canonical TGF-β receptors.

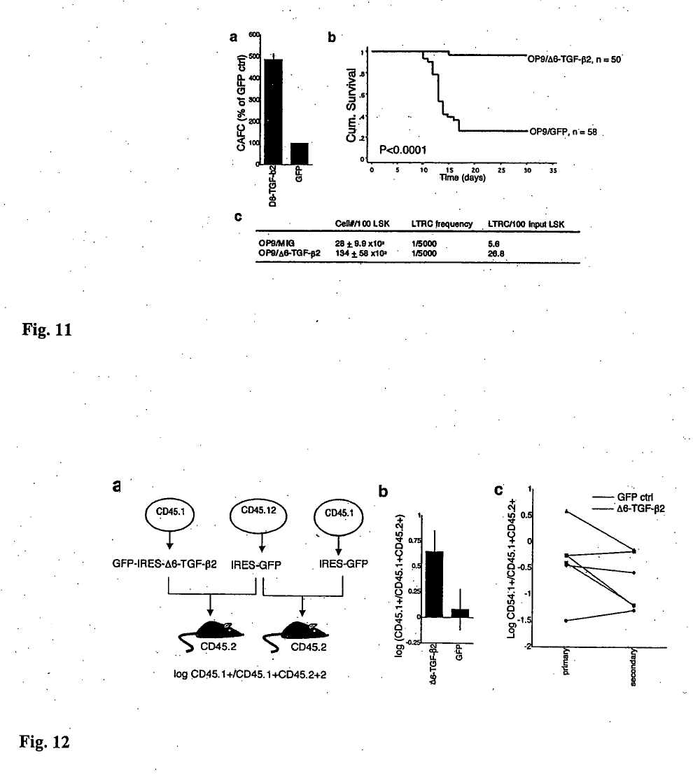

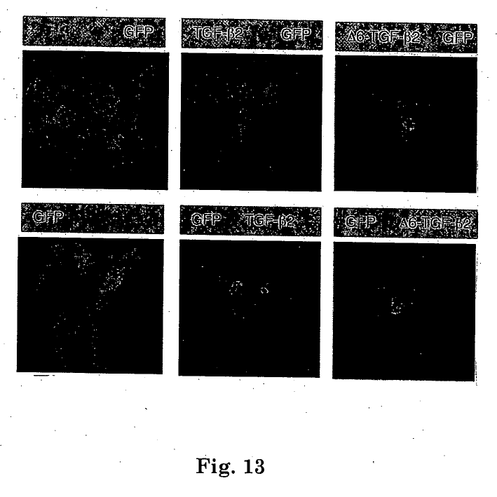

To investigate the intracellular fate of Δ6-TGF-β2, the inventors constructed C- and N-terminal fusions of full length (FL) TGF-β2 and Δ6-TGF-β2 (in C-terminal fusions, GFP is the C-terminus, and in the N-terminal fusions, GFP is the N-terminus of the fusion protein). The C-terminal fusions of both FL and Δ6-TGF-β2 had a similar localization as GFP, i.e. nuclear and cytoplasmic. On the other hand, the N-terminal fusions had a Golgi distribution pattern (FIG. 13). These data indicate similar fates for FL TGF-β2 and Δ6-TGF-β2.

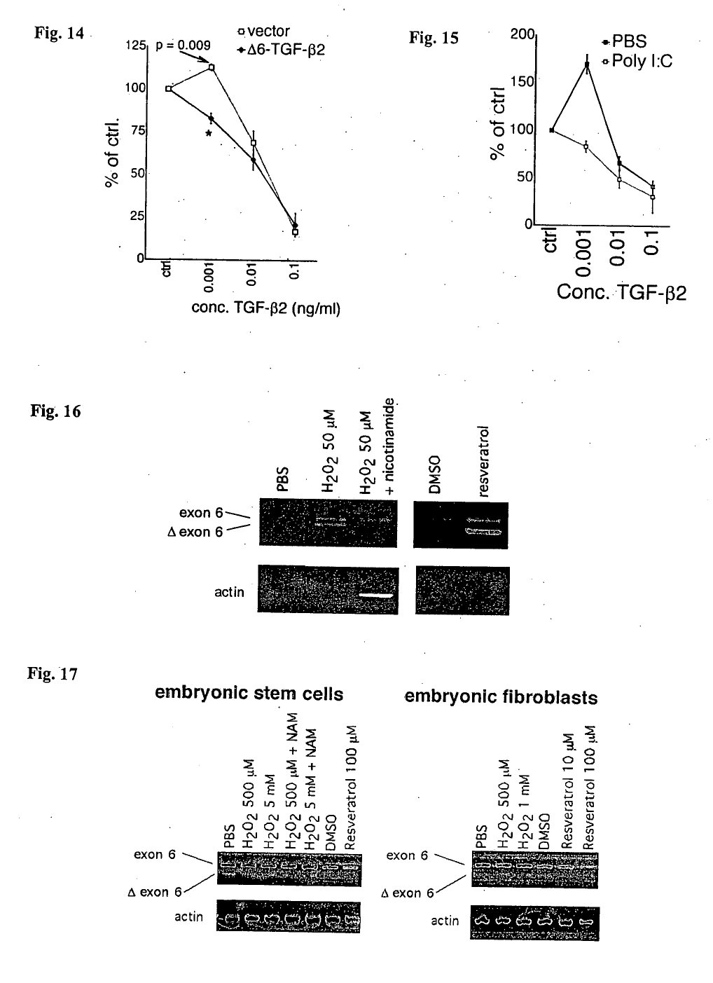

As Δ6-TGF-β2 appeared to be secreted, the inventors tested the effect of recombinant Δ6-TGF-β2-GFP fusion protein on the dose response of TGF-β2 on the proliferation of LSK cells. Addition of the supernatant of 293 cells transfected with Δ6-TGF-β2-GFP blocked the stimulatory effect of TGF-β2 on LSK cell proliferation, whereas supernatant of 239 cells transfected with a control GFP plasmid had no effect (FIG. 14).

Canonical TGF-β2 signaling is initiated by binding of the ligand to the type II TGF receptor (TGF-RII). This ligand-receptor complex then associates with and phosphorylates the type I receptor (TGF-RI), allowing docking and phosphorylation of Smad proteins. To investigate whether the canonical TGF-RII is involved the LSK cell-specific and isoform-specific stimulatory effect of TGF-β2, conditional knockout mice were employed. In these mice, the Tgfr2 gene is flanked by loxP sites, and Cre expression is controlled by the interferon-responsive Mxi promoter. Deletion of the Tgfr2 gene is accomplished by injection of polyI:C. Control mice consisted of floxed Tgfr2^{ft/ft} mice injected with PBS. The stimulatory effect of low concentrations of TGF-β2 was entirely abolished by the deletion of the Tgfr2 gene. However, the inhibitory effect at higher concentrations of TGF-β2 appeared unaffected, indicating the existence of an alternative receptor mediating this part of the TGF-β2 dose response (FIG. 15). Thus, the inventors have now identified four conditions where the LSK-specific stimulatory phase of the TGF-β2 dose responses is abrogated: in the absence of serum, after knockout of TGF-RII, after overexpression of Δ6-TGF-β2, and after addition of exogenous Δ6-TGF-β2. In each of these instances, the inhibitory phase of the TGF-β2 dose response, as well as the dose responses of TGF-β1 and TGF-β3 appeared unaffected.

E. Regulation of Δ6-TGF-β2: Induction by Conserved Stress Response Mechanisms

Δ6-TGF-β2 is induced by stress. TGF-β2 is a positive regulator of HSC cycling, and as Δ6-TGF-β2 appears to block the effects of TGF-β2, it is likely that Δ6-TGF-β2 in fact drives HSC into quiescence during stress. The inventors recognized that is this is a mechanism to protect HSC until the stressful insult is over. In addition Δ6-TGF-β2 is repressed by TGF-β2 signaling, generating a negative feedback loop that may terminate Δ6-TGF-β2 expression when stress has abated.

Stress response mechanisms show a remarkable conservation from yeast to mammals, and are linked to longevity. One of the mechanisms involved in the regulation of longevity and stress responses in lower organisms is Sirt1, the mouse orthologue of yeast SIR2. SIR2 is a NAD-dependent protein deacetylase, which silences DNA in yeast. Due to its NAD-dependence, SIR2 senses the redox state of the cell, and silences DNA, explaining the involvement of SIR2 in life span extension by caloric restriction. In the yeast model, life span extension by nutrient deprivation indeed depends on an intact SIR2 gene. In C. elegans, overexpression of sir2 also extends life span. The closest mouse orthologue of SIR2, SIRT1, deacetylates P53, thereby antagonizing its transcriptional and pro-apoptotic activity. Subsequently, several other proteins have been shown to be deacetylated by Sirt1, including Foxo3a, MyoD and PPARγ. Stress increases the expression and the deacetylase activity of Sirt1, and the actions of Sirt1 tend to prevent apoptosis and enhance maintenance of cells. It is therefore hypothesized that in mammals, Sirt1 prevents stress-induced organ and tissue erosion. Furthermore, the inventors found that Sirt1 expression is high in LSK cells, and near undetectable in lineage+, more mature bone marrow cells (FIG. 17a).

Sirt1 deacetylase activity is blocked by nicotinamide, and induced by resveratrol. Pre-incubation of LSK cells for 1 hour with nicotinamide, abrogated the effect of subsequent addition of H₂O₂ on TGF-β2 pre-mRNA splicing (FIG. 16, left panels). Resveratrol induced pronounced alternative splicing of TGF-β2 pre-mRNA (FIG. 16, right panels).

F. Cell Line Models and Other Cells Types

No induction of Δ6-TGF-β2 by H₂O₂ or by resveratrol was observed in 3T3 cells, mouse embryonic fibroblasts (FIG. 17), embryonic stem (ES) cells (FIG. 17), embryoid bodies, and LSK cells immortalized with Lhx2, although low levels of Δ6-TGF-β2 were detected (not shown). It is of interest that even transformed LSK cells and ES cells (ES cells are tumorigenic in vivo), do not regulate expression of Δ6-TGF-β2. In addition, none of the aforementioned cells showed a stimulatory response to low concentrations of TGF-β2, indicating that the TGF-β2/Δ6-TGF-β2 system described here is specific for normal hematopoietic stem and early progenitor cells. Skin stem cells express low levels of Δ6-TGF-β2. It has been previously published that TGF-β2 enhances the development of hair follicles and that TGF-β2 is expressed in skin stem cells.

G. The Role of Δ6-TGF-β2 in Aging and Longevity

Sirt1 is essential for longevity in several model organisms. Furthermore, it has been widely hypothesized that aging of stem cells may underlie organismal aging, as failing stem cells may cause decreased regeneration of tissues, and therefore, aging of tissues. The inventors have previously shown that quantitative trait loci (QTL) regulating hematopoiesis, including those contributing to LSK frequency and to the response of LSK cells to TGF-β2, and QTL contributing to genetic variation in life span are closely linked at multiple loci, suggesting that the HSC compartment may play a role in organismal aging. In particular, mice with lower numbers of LSK cells tended to live longer. Tgfb2^{+/-} mice have lower numbers of LSK cells, which are more quiescent. It is therefore anticipated that these mice will live longer. The inventors therefore examined whether aging is slowed in Tgfb2^{+/-} mice.

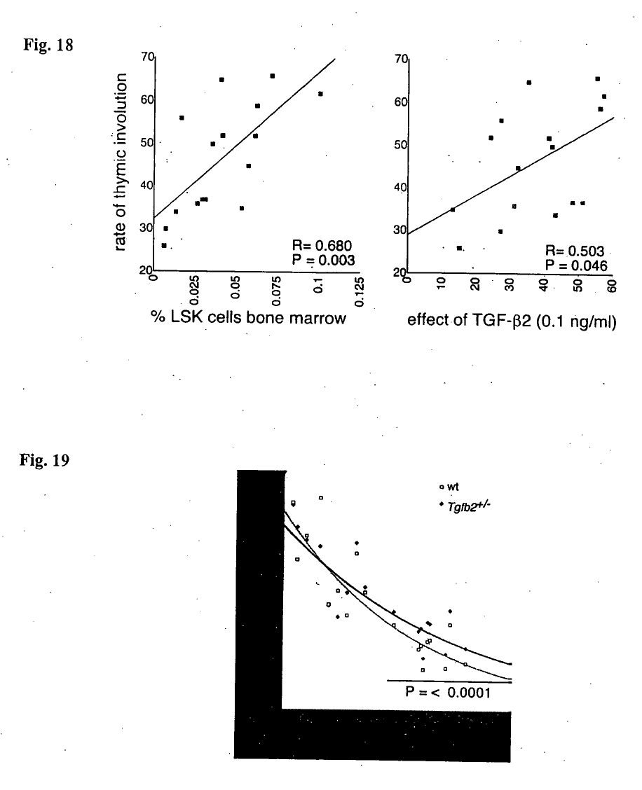

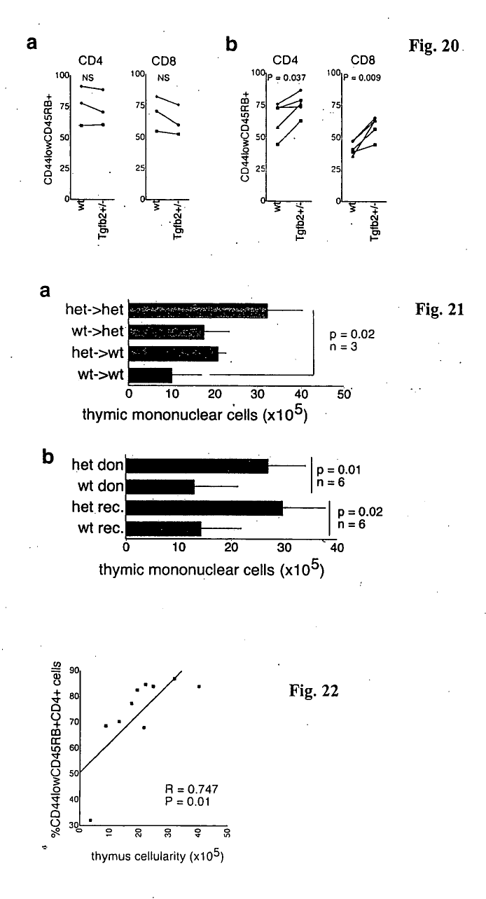

Among genetically different mouse strains (BXD recombinant inbred mice), a correlation was observed between number of LSK cells and their responsiveness to TGF-β2 on one hand, and the rate of thymic involution, a symptom of aging, on the other (FIG. 18). Tgfb2^{+/-} mice have lower numbers of LSK cells, which are more quiescent. Therefore it is anticipated that these mice will live longer. A significant finding in this context is that Tgfb2^{+/-} mice show slower thymic involution and have higher levels of naïve T cells when old (FIG. 19, FIG. 20). Thymic involution and depletion of naïve T cells are considered biomarkers of aging. This effect of TGF-β2 on thymic involution is at least in part dependent on its effect on HSC, as transplantation of Tgfb2^{+/-} bone marrow into wt recipients slows thymic involution (FIG. 21). Note, however, that the Tgfb2 status of the recipient plays a role as well (FIG. 21). The specificity of the TGF-β2 effect for hematopoietic stem cell and progenitor cells, and the correlations shown in FIG. 18, indicate that the effect of the Tgfb2 status of the recipient is also mediated through its effect on transplanted wt HSC. As in the Tgfb2 mice, in the transplanted mice, variation in thymus size clearly had a repercussion on the frequency of naïve T cells (FIG. 22), a biomarker of aging. Taken together, these data indicate that blocking the effect of TGF-β2 on stem cells, and consequent silencing of stem cells, will enhance longevity.

1-7. (canceled)

8. A method of enhancing the transduction of stem cells with a recombinant vector comprising contacting the stem cells undergoing transduction with a functional portion of Δ6-TGF-β2.

9. The method of claim 8 wherein the recombinant vector is a retroviral vector, an adenoviral vector or an adenoassociated viral vector.

10. The method of claim 9 wherein the vector is a lentiviral vector.

11. The method of claim 9 wherein the cells are exposed to the functional portion of Δ6-TGF-β2 ex-vivo during the transduction.

12. A method of inducing quiescence in stem cells comprising contacting stem cells with a functional portion of Δ6-TGF-β2.

13. The method of claim 12 wherein contacting the stem cells comprises contacting a cell within a mammal with a vector operably configured to express the functional portion of Δ6-TGF-β2 from the cell.

14. The method of claim 13 wherein the cell is a hematopoietic cell.

15. (canceled)

16. A method for producing recombinant Δ6-TGF-β2, comprising, expressing a functional portion of Δ6-TGF-β2 in a cell containing a vector operably configured to express the functional portion of Δ6-TGF-β2 from a nucleic acid sequence encoding the functional portion of Δ6-TGF-β2 and obtaining the functional portion of Δ6-TGF-β2 from at least one of the cell line and a media into which the cell line secretes the Δ6-TGF-β2.

17. The method of claim 16 wherein the nucleic acid sequence encoding the functional portion of Δ6-TGF-β2 encodes amino acid sequences selected to secrete the functional portion of Δ6-TGF-β2 from the cell line.

18. An isolated nucleic acid sequence comprising a sequence that encodes a functional portion of Δ6-TGF-β2.

19. A cell line comprising the isolated nucleic acid sequence of claim 18.

20. A composition comprising an isolated functional portion of Δ6-TGF-β2.

21. The composition of claim 20 wherein the isolated functional portion of Δ6-TGF-β2 has the amino acid sequence of SEQ. ID NO. 2.

22. A method for maintaining and expanding hematopoietic stem cells comprising providing the stem cells with conditions for proliferation and contacting the stem cells with Δ6-TGF-β2.

23. The method of claim 22 wherein the stem cells are contacted with Δ6-TGF-β2 by culturing the stem cells in the presence of cells that stably express Δ6-TGF-β2.

24. The method of claim 22 wherein the stem cells are contacted with Δ6-TGF-β2 by transducing the stem cells with a construct that expresses Δ6-TGF-β2.

25. An expanded population of hematopoietic stem cells produced by obtaining hematopoietic stem cells, providing the stem cells with conditions for proliferation and contacting the stem cells with Δ6-TGF-β2.

26. The population of claim 25 wherein the stem cells are contacted with Δ6-TGF-β2 by culturing the stem cells in the presence of cells that stably express Δ6-TGF-β2.

27. The population of claim 25 wherein the stem cells are contacted with Δ6-TGF-β2 by transducing the stem cells with a construct that expresses Δ6-TGF-β2.
